(19) United States
(12) Patent Application Publication
(10) Pub. No.: US 2013/0303938 A1
(43) Pub. Date: Nov. 14, 2013

(54) SPEED AND DISTANCE ESTIMATION TEST DEVICE

(71) Applicant: COSKUNOZ HOLDING ANONIM SIRKETI, (US)

(72) Inventors: Remzi Sulo, Bursa (TR); Gulcer Aydin, Bursa (TR)

(73) Assignee: COSKUNOZ HOLDING ANONIM SIRKETI, Bursa (TR)

(21) Appl. No.: 13/834,176

(22) Filed: Mar. 15, 2013

(30) Foreign Application Priority Data
Nov. 4, 2011 (EP) PCT/EP2011/069394

(51) Int. Cl. A61B 3/02; A61B 3/00

(52) U.S. Cl. CPC A61B 3/02; A61B 3/0033; A61B 3/0041
USPC 600/558

(57) ABSTRACT

The present invention relates to a test device which is designed for measuring the caution and intuitional capability of the individuals, by providing the individual to make an estimate about the position of the stimulus (U1) at a certain time after the visual stimulus (U1) disappears from the screen (20) and after the stimulus continues the movement thereof in an invisible manner.

### CROSS-REFERENCE TO RELATED APPLICATION

This is a Continuation application of PCT Patent Application PCT/EP2011/069394 filed on Nov. 2, 2011, which claims the priority benefit of Turkish Patent Application Serial No. 2010/09701, filed Nov. 25, 2010, which is hereby incorporated herein by reference.

### TECHNICAL FIELD

The present invention relates to a test device which measures individuals' correctness, caution and intuitional capability in the estimation of the required time duration for the stimulus to reach the desired point or in the estimation of the position of the stimulus on the screen after the visual stimulus disappears at a predetermined point on the screen taking into consideration that the visual stimulus moves at a certain speed after it disappears from the screen.

### BACKGROUND

Industrial and Organizational Psychology is a psychology science which was applied in the industrial plants in the past and which is applied today in pluralities of foundations. When Industrial and Organizational Psychology is examined, it is observed that Industrial and Organizational Psychology has two basic structures. The first of these structures is the harmonization of the individual to the work and the other is the harmonization of the work to the individual. The harmonization of the individual to the work comprises the steps of personnel selection, personnel training and the professional help; on the other hand, the harmonization of the work to the individual comprises the processes of task design, the arrangement of the tools according to the individual, and shaping the working conditions according to the physical and psychological structure of the individual.

In the area of Industrial and Organizational Psychology, pluralities of measurement and evaluation tools are used for the selection of the suitable personnel. Psycho-technical evaluation can be defined as the measurement of the physical, psychomotor and cognitive abilities and the personal characteristics of an individual by means of pluralities of tests which are linguistic or non-linguistic. In the area of Industrial and Organizational Psychology, in personnel selection, in other words, in the selection of the suitable applicant to the suitable position, psycho-technical evaluation is used. The physical power (like the muscle power, body power), psychomotor (like coordination ability, response speed) and cognitive abilities (like judgment, visual and auditory attention and speed distance forecast) and the personal abilities (like willingness to take risks, anxiety level) are measured and the best applicant is selected in terms of the abilities which the task requires.

Pluralities of studies are realized for measuring psychomotor abilities and some techniques are determined.

The patent application U.S. Pat. No. 4,383,827 relates to a driving simulator and it comprises all of the control fixtures which are required for a vehicle. During the simulation, the visual members related to traffic are formed on the screen, and at the predetermined points of the test, the drive responses, which the individuals give during the problems occurring on the screen, are recorded.

In the patent application WO 2006031131, the object is to record the responses of the individuals during the problems appearing on the screen under different air and road conditions during driving inside the simulator.

In the patent application US 2010167246, a test is applied where the next positions of the visual member are requested to be followed or estimated by the individuals existing inside a simulator having more than one monitor during the passage of the visual members to the next screen, in response to the visual members which are simultaneously viewed on said screens.

As a result, in the related technical field, a test device is not disclosed which measures the position of a moving visual member after certain time duration or which measures the time duration it takes to reach a determined target.

### SUMMARY THE INVENTION

The present invention is a test device which measures the psycho-technical abilities, in order to bring new advantages to the related technical field.

The main object of the present invention is to measure the caution and intuitional capability of the individual, by requesting the individual to estimate the position of the stimulus on the screen after certain time duration as a result of the stimulus disappearing from the screen and advancing at a constant speed.

Another object of the present invention is to measure the correctness ratio in the intuitive calculation of the passed time duration by the individual as a result of the reaction of the individual when the individual thinks that the stimulus reaches the desired point after the stimulus disappears from the screen.

In order to realize all of the abovementioned objects and the objects which are to be obtained from the detailed description below, the present invention is a test device which is designed for measuring the caution and intuitional capability of the individuals, by providing the individual to make an estimate about the position of the stimulus at a certain time after the stimulus disappears from the screen and after the stimulus continues the movement thereof in an invisible manner, characterized by comprising:
a. An operating system,
b. At least one input unit with a key pad thereon, in order to provide the data entry to the operating system,
c. One image source which is related to the operating system and which comprises at least one path, at least one stimulus and at least one station which are different from each other,
d. At least one data source which is related to the operating system, which comprises the data therein which is entered by means of the input unit and which comprises the test data to be applied,
e. At least one record unit which is related to the operating system and which keeps the answers given by the individuals during the test,
f. At least one screen which is related to the operating system, which displays said paths, the stimulus and the station and which provides the individuals to realize the selections thereof and which is a touch-screen,
g. A button which is related to the operating system and which provides the individuals to realize the estimation thereof when the individuals think that the stimulus reaches the desired station according to the movement of the stimulus during the test,
h. At least one timer which is related to the operating system and which measures the beginning, finishing and process duration for each process on the screen during test steps,
i. A processor unit which is related to the operating system, which provides the displaying of at least said paths, the stimulus and the station on the screen at a predetermined arrangement and which functions together with said timer and which provides the recording of the target station number, station number where the individual gives response, the time duration which the stimulus has to exist in the determined station, the time duration which the individual gives response to the stimulus and which provides these items to be recorded to the record unit.

In another preferred embodiment of the subject matter invention, the responses of the individuals which are mentioned in step (i) and which are recorded by the record unit are the following: the deviation level and the deviation direction from the target station in the destination estimation part; the number of responses beyond the target station; the number of responses on the target station; the number of responses which are behind the target station; the total response duration; the average response duration; the target station number in the time of arrival test; the required time duration; the response duration; the deviation time duration from the correct time; the number of responses which are given at a late time; the number of responses which are given in time; the number of responses which are given at an early time.

In another preferred embodiment of the subject matter invention, in said step (i), according to the predetermined arrangement; the path, the movement speed of the stimulus and the station to be active appear on the screen; these are waited on the screen in order for the individual to realize an estimation; afterwards, again another group comprising the path, the movement speed of the stimulus and the station to be active appears on the screen and these continue at a predetermined sequence.

In another preferred embodiment of the subject matter invention, there is at least one compiling unit which is related to the operating system and which classifies the data recorded by the record unit.

In another preferred embodiment of the subject matter invention, there is an analysis unit which is related to the operating system and which provides the comparison of the files recorded by the record unit.

In another preferred embodiment of the subject matter invention, there is at least one ear phone which provides the transferring of the voiced instructions to the individuals during the test.

In another preferred embodiment of the subject matter invention, said operating system is a computer.

In another preferred embodiment of the subject matter invention, said input unit is a keyboard.

In another preferred embodiment of the subject matter invention, said image source, data source and record unit are hard disks.

In another preferred embodiment of the subject matter invention, there is a platform whereon all of the abovementioned members are placed at a predetermined arrangement and on which the individuals to whom the test is applied are standing.

In another preferred embodiment of the subject matter invention, there is at least one plate whereon the individuals can place their hands on the platform and which provides the individuals to stay in the suitable position during the test.

In another preferred embodiment of the subject matter invention, there is at least one button which provides the given response to be mentioned, in order to be used in the arrival test of the individuals on the platform.

In another preferred embodiment of the subject matter invention, the difference between the first path and the second path is that the paths have different rotational bending.
a. In another preferred embodiment of the subject matter invention, the stimulus is in the form of an arrow.

A test method which is designed for measuring the caution and intuitional capability of the individuals, by providing the individual to make an estimate about the position of the stimulus at a certain time after the stimulus disappears from the screen and after the stimulus continues the movement thereof in an invisible manner, characterized by comprising the steps of:
a. Recording of the individual to the operating system,
b. Starting the test from the data source in the direction of the predetermined timings in relation to the processor unit timer,
c. The operating system displays the first path on the screen where there are more than one station on said first path,
d. The stimulus moves inside the first path under predetermined conditions,
e. The first path waits on the screen for certain duration in order for the individual to give response,
f. The response (the preferred station or no response) of the individual is recorded by the record unit,
g. The second path, which has more than one station, is displayed on the screen by the operating system,
h. The stimulus moves inside the second path under predetermined conditions,
i. The second path waits on the screen for certain duration in order for the individual to give response,
j. The response (the preferred station or no response) of the individual is recorded by the record unit,
   Said test method also comprises the following steps:
k. The first path and the second path appear on the screen one after the other at a predetermined arrangement,
l. The stimulus moves inside the first path and the second path one after the other under predetermined conditions,
m. The responses like station selection which the individual gives against all of the paths or no response is recorded by the record unit,
   Said test method also comprises the following steps:
n. The operating system displays the first path on the screen, where the first path comprises more than one station,
o. The stimulus moves inside the first path under predetermined conditions,
p. The first path waits on the screen for certain duration in order for the individual to be able to press to the button on the platform,
q. The response (the duration of pressing to the button when the individual estimates that the stimulus reaches the target station) of the individual is recorded by the record unit,
r. The second path, which has more than one station, is displayed on the screen by the operating system,
s. The stimulus moves inside the second path under predetermined conditions,
t. The second path waits on the screen for certain duration in order for the individual to give response,
u. The response (the duration of pressing to the button when the individual estimates that the stimulus reaches the target station) of the individual is recorded by the record unit
   Said test method also comprises the following steps:
v. The first path and the second path appear on the screen one after the other at a predetermined arrangement,
w. The stimulus moves inside the first path and the second path one after the other under predetermined conditions,
x. The responses (the duration of pressing to the button when the individual estimates that the stimulus reaches the target station) like station selection or no response is recorded by the record unit,
y. The test ends.

In order for the embodiment and the advantages of the subject matter invention to be understood in the best manner with the additional elements, it has to be evaluated with the figures explained below.

### BRIEF DESCRIPTION OF THE FIGURES

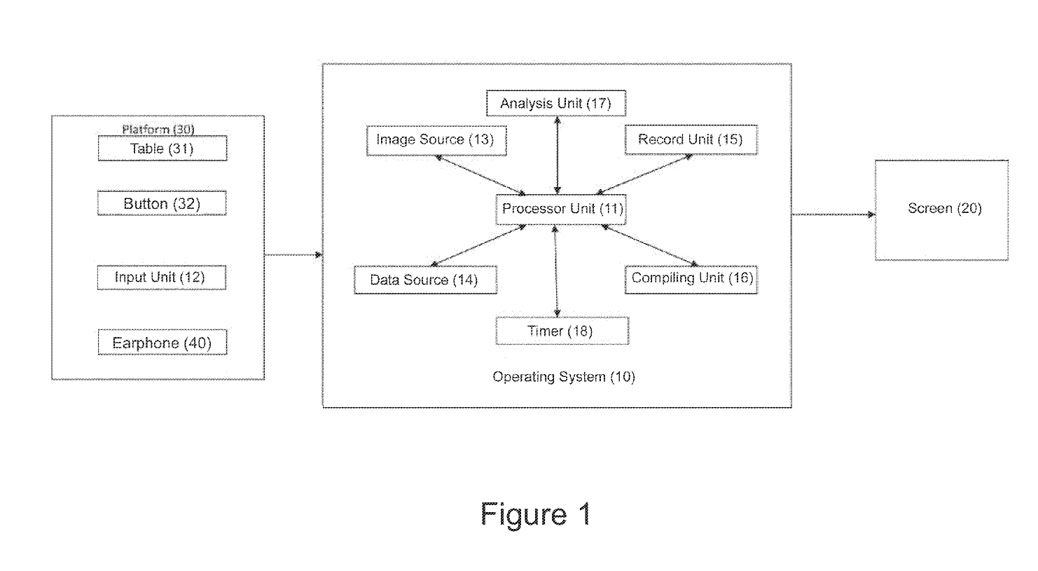

In FIG. 1, the schematic view of the device is given.

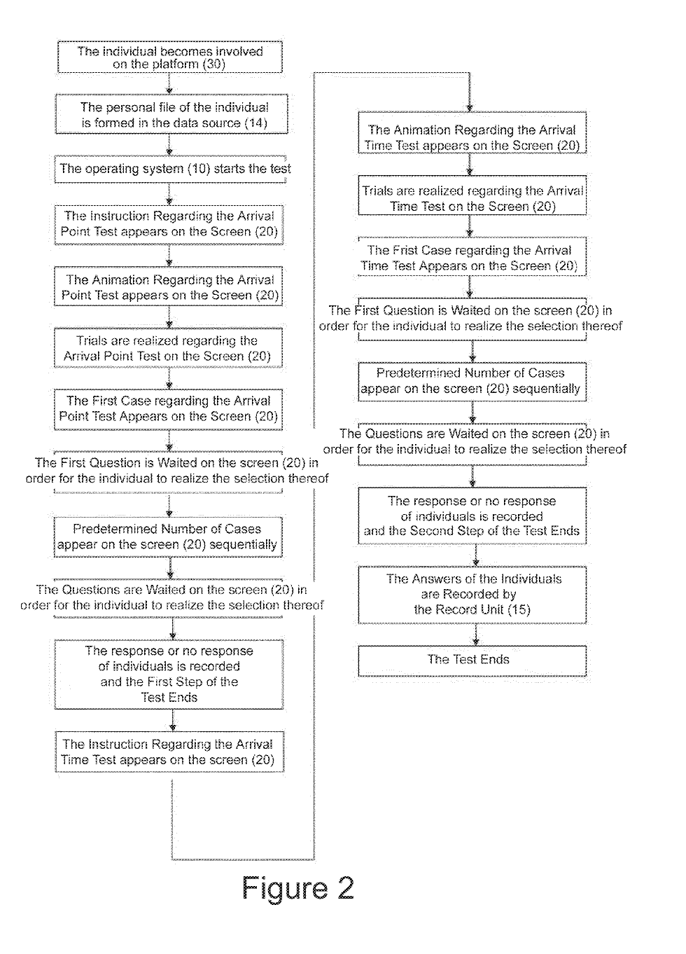

In FIG. 2, the flow schema of the test is given.

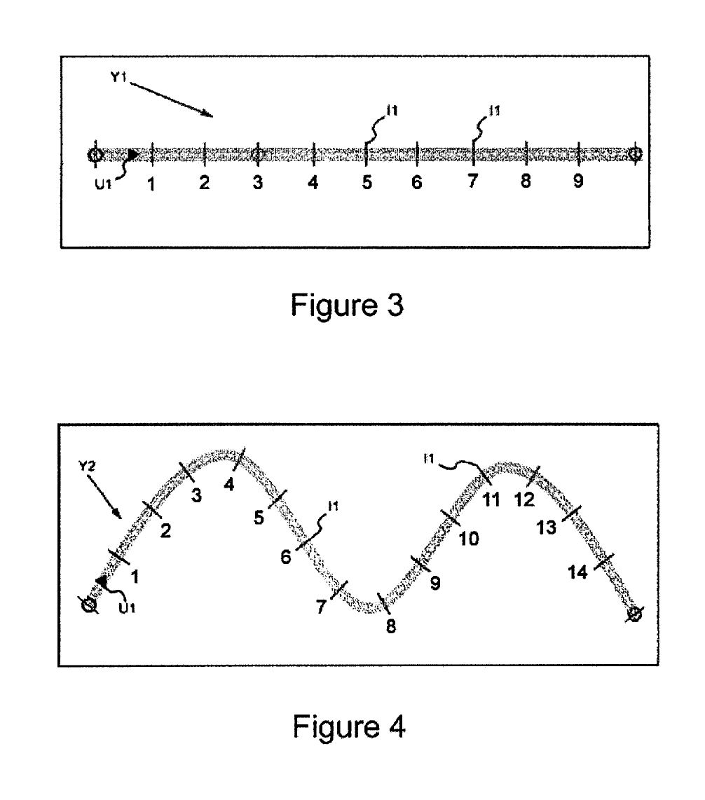

In FIG. 3, the representative view of the first path is given.

In FIG. 4, the representative view of the second path is given.

### DETAILED DESCRIPTION OF THE INVENTION

In this detailed description, the subject matter system is explained with references to examples without forming any restrictive effect in order to make the subject more understandable. Accordingly, in the detailed description and in the figures below, the subject matter invention relates to a test device which is designed for measuring the caution and intuitional capability of the individuals, by providing the individual to make an estimate about the position of the stimulus at a certain time after the stimulus disappears from the screen and after the stimulus continues the movement thereof in an invisible manner, in the area of measurement and evaluation of the psychomotor and cognitive processes.

As can be seen in FIG. 1, the schematic view of the subject matter device is given. The device essentially comprises an operating system (10); a screen (20) which visually illustrates the data related to the operating system (10); a platform (30) where the individual is subject to test; and an earphone (40).

The operating system (10) has a structure similar to a computer frame, it has housings or slots (not illustrated in the figure) to which the other members can be connected. Moreover, around the slots for protecting said members, there are structures similar to protective plate (not illustrated in the figure). There is an input unit (12) which is related to the operating system (10) and whereon the key pad is placed. There is an image source (13), a data source (14), a record unit (15), a compiling unit (16), an analysis unit (17) and a timer (18), related to said slots inside the operating system (10). Moreover, there is a processor unit (11) which is positioned inside the operating system (10).

On the platform (30), there are two plates (31) which are designed to be suitable to the ergonomic form of human hand. Moreover, there is a button (32) on the platform (30). The individuals place their hands on said plates (31) and they take the ready position for answering the questions.

The operation of the members existing inside the operating system (10) is as follows. By means of the keys on said input unit (12), data input can be realized to the operating system (10). Said data can be the personal data of the individuals who are subject to test, they provide the realization of all of the data input like the connections and relations between the members related to the test. The data whose input is realized is kept and stored in the data source (14). The stored data can be viewed again and they can be used again and when required, changes can be realized thereon. The image source (13) embodies the data which is to be used visually. The data existing inside the data source (14) and the visual data existing inside the image source (13) are related and thereby the relations between the data are realized. The record unit (15) records the personal information of the individuals subject to the test and it records the answers of the individuals during the test. Thanks to this, the record unit (15) keeps the information of the numerical data like how many situations an individual faces in the personal data file during test, how the individual responses to the situation he/she faces, how long the response the individual gives takes. The compiling unit (16) provides the classification of the information said record unit (15) records when there is more than one individual. When the information of the individuals who are tested in this manner is desired to be accessed, the access is facilitated in the frame of a certain classification. The analysis unit (17) provides the comparison of the test results of more than one individual. The analysis unit (17) numerically analyses the test results of the selected people when the test results recorded by the record unit (15) is desired to be compared, and the analysis unit (17) provides the advantages and disadvantages between the individuals to be taken into consideration. The analysis unit (17) provides the sequencing of the results in addition to the comparison process. The processor unit (11) has the task of matching the data buses between the members. The timer (18) has the task of measuring the durations predetermined for the test; measuring the passage duration between the cases; and measuring the time difference between the time when the individuals face the stimulus and the time when they give response to the stimulus. The timer (18) functions together with the processor unit (11).

On the screen (20), the data coming from the operating system (10) is transformed into visual form. Said screen (20) is a touch-screen and provides the individuals to realize their selections by touching the screen (20). Inside the image source, there are two different paths called the first path (Y1) and the second path (Y2) and inside the image source, there is the figure of the moving stimulus (U1). Moreover, there is a station (I1) figure inside the image source (13). Said first path (Y1) is a flat path and there are nine stations (I1) on the first path (Y1) except the beginning and the finishing stations (I1). Said second path (Y2) is a path with bending and there are fourteen stations (I1) on the second path (Y2) except the beginning and finishing stations (I1). The stations (I1) have a square form and there are the numbers on the stations (I1) mentioning the station (I1) number. The moving stimulus (U1) has an arrow form with a size so as to move inside the first and the second path (Y1, Y2) and so as to be easily detected visually by the individuals.

The test to be applied to the individuals comprises two steps. In the first step, there are two parts, in the first part, the individual is requested to estimate the station which the visual stimulus (U1) arrives after the visual stimulus (U1) disappears from the screen taking into consideration that the visual stimulus (U1) moves with the same constant speed in an invisible manner; and in the second step, the individual is requested to estimate the time which the visual stimulus (U1) is considered to reach the predetermined station (I1) after the visual stimulus (U1) disappears from the screen taking into consideration that the visual stimulus (U1) moves with the same constant speed in an invisible manner. From the first path (Y1) and the second path (Y2) figures which are to be used in the first part, four of the nine stations (I1) on the first path (Y1) are target stations (I1); and nine of the fourteen stations (I1) on the second path (Y2) are target stations (I1). The stimulus (U1) can pass through these paths with four different movement speeds. However, during the situation on the screen, there is no speed change after the stimulus disappears from the screen (20), the visual stimulus moves at constant speed. In both test steps, the stimulus (U1) disappears from the screen (20) at the third station (I1) on the paths (Y1, Y2). For the first step of the test, a warning message comes from the ear phone (40) after predetermined time duration within each question. In the second step of the test, a light flashes on the station (I1) where the arrival time is requested to be estimated. If the individuals do not place their hands on the plates (31) existing on the platform (30), the system does not pass to the next question.

The operation of the test device is as follows. The personal information of the individual to be tested is recorded to the record unit (15) with the help of the input unit (12). The individual wears the ear phones (40) and he/she begins applying the instructions appearing on the screen (20). The instructional writings appear on the screen (20) on a gray background. The instruction writings, appearing on the screen (20), are announced to the individual by means of the ear phone (40). In the instructions, comprehensive and detailed information is given to the individual about the things the individual should do in the first step of the test; about how he/she should stand on the platform; about general information about the first step of the test; about the signal tone coming from the ear phone; and about how the individual should realize selection. Moreover, after the required information is given, an animation, showing how the individuals should behave in the first step of the test, is illustrated on the screen (20). After the animation finishes, the instructions, informing that some trials will be realized, appear on the screen (20). The first step of the test begins after two trials finish and after the answers are given to the two trials. In the first step of the test, the individuals are requested to select the station (I1) number, where they think that the visual stimulus (U1) reaches as it moves in the same constant speed after it disappears from the screen, by touching on the screen (20). After the individual realizes his/her selection, if his/her selection is correct, “your estimation is correct” instruction appears on the screen (20). If the individual's selection is wrong, “your selection is wrong; when you heard the signal tone, the stimulus arrived station (I1) x” instruction appears on the screen (20). If the individual does not realize selection in ten seconds, “you did not realize selection; when you heard the signal tone, the stimulus arrived station (I1) x” instruction appears on the screen (20). If the individual does not place both of his/her hands on the plate (31), “please place your hands on the plates (31)” instruction appears on the screen (20) after five seconds. The first step of the test finishes after a predetermined number of cases are realized. In each case, two different paths (Y1, Y2), four different speeds at which the stimulus (U1) moves and different station (I1) combinations are used.

After the arrival place estimation, which is the first step of the test, finishes, on the screen (20), the instructions regarding the arrival time appears on the screen which is the second part of the test. After the detailed instruction regarding the test is announced to the individuals, an animation, showing how the individuals should behave in the second step of the test, is illustrated on the screen (20). After the animation finishes, the instructions, informing that some trials will be realized, appear on the screen (20). After two trials are realized and after the required answers are given to these trials, the second step of the test begins. In the arrival point estimation test, the individuals are requested to press the button (32) in the front thereof when they think that the stimulus (U1) arrives at the target station (I1) after the stimulus (U1) moves at the same speed after the stimulus (U1) disappears from the screen (20). In each question, after the stimulus (U1) begins moving and when the stimulus (U1) arrives at the third station (I1), it disappears from the screen (20) and it continues moving with the same speed. When the individual thinks that the stimulus (U1) arrives at the target station (I1), he/she presses the button (32) and thereby he/she realizes estimation. If the estimation is correct, on the screen (20), “your selection is correct” message appears. If the individual presses the button (32) early or late, on the screen (20), “you pressed the button (32) early or you pressed the button (32) late and when you pressed the button (32), the stimulus (U1) has reached the illustrated point on the path (Y1, Y2)”. If the individual does not realize estimation within ten seconds after the stimulus (U1) passes the target station (I1), on the screen (20), “you did not realize estimation” instruction appears. Since the probability of the individuals to realize the correct estimation about the arrival point is low, the system is adjusted so that the estimation of the individual is correct within one quarter of the distance between the target station (I1) and the station (I1) next to the target station (I1). After the individuals realize the estimations thereof, if they do not place their hands on the plates (31) within five seconds, the “please place your hands on the plates (31)” instruction appears on the screen (20). The second step of the test finishes after predetermined number of cases are realized. In each question, two different paths (Y1, Y2), four different speeds of the stimulus (U1) and different active stations (I1) are used.

During the test, the behaviors of the individuals are recorded by the record unit (15). In the arrival point test, which is the first step of the test, the recorded items are the following: the target station (I1) number; the number of the station (I1) which the individual gives response; the response time; the deviation direction and level from the target station (I1); the number of responses which are given beyond the target station (I1); the number of responses given above the target station (I1); the number of responses given before the target station (I1); the total response duration and the average response duration. In the arrival time test, which is the second step of the test, the recorded items are the following: the target station (I1) number; the required time duration; the response duration; the time deviation; the number of responses which are given late; the number of responses which are given in time; the number of responses which are given early; the total number of responses.

The protection scope of the present invention is set forth in the annexed Claims and cannot be restricted to the illustrative disclosures given above, under the detailed description. It is because a person skilled in the relevant art can obviously produce similar embodiments under the light of the foregoing disclosures, without departing from the main principles of the present invention.

1. A test device which is designed for measuring the caution and intuitional capability of the individuals, by providing the individual to make an estimate about the position of the stimulus at a certain time after the visual stimulus disappears from the screen and after the stimulus continues the movement thereof in an invisible manner, characterized by comprising:
an operating system,
at least one input unit with a key pad thereon, in order to provide the data entry to the operating system,
an image source which is related to the operating system and which comprises at least one path, at least one stimulus and at least one station which are different from each other,
at least one data source which is related to the operating system, which comprises the data therein which is entered by means of the input unit and which comprises the test data to be applied,
at least one record unit which is related to the operating system and which keeps the answers given by the individuals during the test,
at least one screen which is related to the operating system, which displays said paths, the stimulus and the station and which provides the individuals to realize the selections thereof and which is a touch-screen,
a button which is related to the operating system and which provides the individuals to realize the estimation thereof when the individuals think that the stimulus reaches the desired station according to the movement of the stimulus during the test,
at least one timer which is related to the operating system and which measures the beginning, finishing and process duration for each process on the screen during test steps, and
a processor unit which is related to the operating system, which provides the displaying of at least said paths, the stimulus and the station on the screen at a predetermined arrangement and which functions together with said timer and which provides the recording of the target station number, station number where the individual gives response, the time duration which the stimulus has to exist in the determined station, the time duration which the individual gives response to the stimulus and which provides these items to be recorded to the record unit.

2. A test device according to claim 1, characterized in that the responses of the individuals recorded by the record unit are the following: the deviation level and the deviation direction from the target station in the destination estimation part;
the number of responses beyond the target station; the number of responses on the target station; the number of responses which are behind the target station;
the total response duration; the average response duration; the target station number in the time of arrival test; the required time duration; the response duration; the deviation time duration from the correct time; the number of responses which are given at a late time; the number of responses which are given in time; the number of responses which are given at an early time.

3. A test device according to claim 1, characterized in that according to the predetermined arrangement; the path, the movement speed of the stimulus and the station to be active appear on the screen; these are waited on the screen in order for the individual to realize an estimation; afterwards, again another group comprising the path, the movement speed of the stimulus and the station to be active appears on the screen and these continue at a predetermined sequence.

4. A test device according to claim 1, characterized in that there is at least one compiling unit which is related to the operating system and which classifies the data recorded by the record unit.

5. A test device according to claim 1, characterized in that there is an analysis unit which is related to the operating system and which provides the comparison of the files recorded by the record unit.

6. A test device according to claim 1, characterized in that there is at least one ear phone which provides the transferring of the voiced instructions to the individuals during the test.

7. A test device according to claim 1, characterized in that said operating system is a computer, said input unit is a keyboard and said image source, data source and record unit are hard disks.

8. A test device according to claim 1, characterized in that there is a platform whereon all of the abovementioned members are placed at a predetermined arrangement and on which the individuals to whom the test is applied are standing.

9. A test device according to claim 8, characterized in that there is at least one plate whereon the individuals can place their hands on the platform and which provides the individuals to stay in the suitable position during the test.

10. A test device according to claim 1, characterized in that there is at least one button which provides the given response to be mentioned, in order to be used in the arrival test of the individuals on the platform.

11. A test device according to claim 1, characterized in that the difference between the first path and the second path is that the paths have different rotational bending.

12. A test device according to claim 1, characterized in that the stimulus is in the form of an arrow.

13. A test method which is designed for measuring the caution and intuitional capability of the individuals, by providing the individual to make an estimate about the position of the visual stimulus at a certain time after the stimulus disappears from the screen and after the stimulus continues the movement thereof in an invisible manner, characterized by comprising the steps of:
recording of the individual to the operating system,
starting the test from the data source in the direction of the predetermined timings in relation to the processor unit timer,
the operating system displays the first path on the screen where there are more than one station on said first path,
the stimulus moves inside the first path under predetermined conditions,
the first path waits on the screen for certain duration in order for the individual to give response,
the response (the preferred station or no response) of the individual is recorded by the record unit,
the second path, which has more than one station, is displayed on the screen by the operating system,
the stimulus moves inside the second path under predetermined conditions,
the second path waits on the screen for certain duration in order for the individual to give response, and
the response (the preferred station or no response) of the individual is recorded by the record unit.

14. A test method according to claim 13, characterized by comprising the steps of:
the first path and the second path appear on the screen one after the other at a predetermined arrangement,
the stimulus moves inside the first path and the second path one after the other under predetermined conditions,
the responses like station selection which the individual gives against all of the paths or no response is recorded by the record unit.

15. A test method according to claim 13, characterized by comprising the steps of:
the operating system displays the first path on the screen, where the first path comprises more than one station,
the stimulus moves inside the first path under predetermined conditions,
the first path waits on the screen for certain duration in order for the individual to be able to press to the button on the platform,
the response (the duration of pressing to the button when the individual estimates that the stimulus reaches the target station) of the individual is recorded by the record unit,
the second path, which has more than one station, is displayed on the screen by the operating system,
the stimulus moves inside the second path under predetermined conditions,
the second path waits on the screen for certain duration in order for the individual to press the button on the platform, and
the response (the duration of pressing to the button when the individual estimates that the stimulus reaches the target station) of the individual is recorded by the record unit.

16. A test method according to claim 13, characterized by comprising the steps of:
the first path and the second path appear on the screen one after the other at a predetermined arrangement,
the stimulus moves inside the first path and the second path one after the other under predetermined conditions,
the response (the duration of pressing to the button when the individual estimates that the stimulus reaches the target station) of the individual or no response is recorded by the record unit, and
the test ends.

17. A test method according to claim 13, characterized in that the responses of the individuals which are recorded by the record unit are selected from the following: the number of the target station in the arrival point estimation part; the station number where response is given; the response duration; the deviation level and deviation direction from the target station; the number of responses given beyond the target station; the number of responses on the target station; the number of responses which are behind the target station; the total response duration; the average response duration; the target station number in the time of arrival test; the required time duration; the response duration; the deviation time duration from the correct time; the number of responses which are given at a late time; the number of responses which are given in time; the number of responses which are given at an early time.

18. A test method according to claim 13, characterized in that said operating system is a computer.

19. A test method according to claim 13, characterized in that said record unit is a hard disc.

20. A test method according to claim 13, characterized in that the difference between the first path and the second path is that the paths have different rotational bending.
